(19) United States
(12) Patent Application Publication
(10) Pub. No.: US 2013/0053728 A1
(43) Pub. Date: Feb. 28, 2013

(54) SYSTEMS AND METHODS RELATED TO WASTE DISCHARGING FOR AIDING PATIENTS CONCERNING PROSTATE IRREGULARITIES

(71) Applicant: MAN LOK, Hong Kong (HK)

(72) Inventor: MAN LOK, Hong Kong (HK)

(21) Appl. No.: 13/217,661

(22) Filed: Aug. 25, 2011

(51) Int. Cl. A61B 5/20

(52) U.S. Cl. 600/573

(57) ABSTRACT

Systems and methods are provided concerning waste discharging for aiding patients concerning prostate irregularities. According to one embodiment, this invention includes a waste discharging system for aiding patients concerning prostate irregularities comprising a toilet shared by many persons, a detector detecting that urination occurs, a RFID reader attached to the toilet, a RFID tag associated with a person with prostate concerns, a urine collector attached to the toilet, a pressure sensor associated with the toilet, a timer associated with the toilet, an IR transceiver associated with the toilet, an IR transceiver associated with the door; and a camera outside a door of a room containing the toilet, wherein the camera is used to capture identities of persons detected with prostate irregularities but without associating with RFID tag.

### FIELD OF THE INVENTION

This invention is generally related to discharging waste for aiding patients concerning prostate irregularities. Specifically, this invention detects whether a person urinating is the person with prostate concerns, and if the person is indeed detected with urinating problems, the inventive system reports the problems to associated medical centers for immediate attentions.

### BACKGROUND OF THE INVENTION

According to many studies, one in four men will be troubled to some degree by enlargement of the prostate gland. By the time they reach 80 most men have prostate-related concerns. Prostate Health issues have become an area of great concern for men. Many are forced to get up several times at night to go to the toilet. Due to the irritation, and sometimes embarrassment, a good portion of men have refused to seek medical help regarding aging prostate problems. Since prostate has become a wide spread problem for aging men and has received a lot of attention from the medical world, there is a need to raise the alarms for men who suffer from weakening prostate glands. Men suffering from prostate enlargement have difficulty in urinating. In the early stage, males with prostate problems include difficulty passing urine and weak irregular streams. These people tend to have to get up throughout the night to pass water. Some of which even need go to toilets urgently without much prior notice. They may feel that the bladder isn't empty and continually needing to go to the toilet. Many of them are frustrated with the duration of urinating. Worse, many found dribbling after passing urine. To many men, they may think that as they get grow older, they have no choice but to suffer the irritating and embarrassing problems a growing prostate gland brings. However, it has been known that they're mistaken. They don't have to spend their nights in and out of the toilet and their days developing a knowledge of local lavatories that betters any tourist guide. Treatment is available for the most common cause of this problem, and many of the treatments do not always involve an operation. Therefore, when signs concerning prostate deteriorations are found, the men associated thereof should be alerted promptly, and proper medical treatment should be provided accordingly.

### SUMMARY OF THE INVENTION

According to embodiments of the invention, a waste discharging system for aiding patients concerning prostate irregularities is provided. The system comprises a toilet shared by many persons; a detector detecting that urination occurs; a RFID reader attached to the toilet; a RFID tag associated with a person with prostate concerns; a urine collector attached to the toilet; a pressure sensor associated with the toilet; a timer associated with the toilet; an IR transceiver associated with the toilet; an IR transceiver associated with the door; a camera outside a door of a room containing the toilet, wherein the camera is used to capture identities of persons detected with prostate irregularities but without associating with RFID tag; a processor; and a memory storing instructions that cause the processor to execute a method.

According to one aspect of the invention, the inventive method comprises detecting whether a person urinating is the person with prostate concerns, based on the RFID reader attached to the toilet and the RFID tag associated with the person with prostate concerns; if the person is the person with prostate concerns: determining whether the person has total blockage of urine by using the urine collector attached to the toilet, the pressure sensor, the timer, and the IR transceiver associated with the toilet, in light of measurements, including: frequency of the urination, duration of the urination, volume of the urination, and the pressure of urination; and if it is determined that the total blockage of urine occurs, reporting to a medical center by electronic means, wherein the report contains the measurements with respect to the person with prostate concerns; and if the person is a person other than the person with prostate concerns: determining whether the person has total blockage of urine, in light of measurements, including: frequency of the urination, duration of the urination, volume of the urination, and pressure of urination; and if it is determined that the total blockage of urine occurs, using the camera to capture the identities of the person urinating when the person is leaving the room containing the toilet upon detected by the IR transceiver associated with the door, so that the person can be notified for having prostate irregularities.

In another aspect of the invention, the method comprises detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns; if the person is the person with prostate concerns: determining whether the person is subject to chronic retention of urination, in light of measurements, including: frequency of the urination, duration of the urination, volume of the urination, and the pressure of urination; and if it is determined that the person is subject to chronic retention of urination, reporting to a medical center by electronic means, wherein the report contains the measurements with respect to the person with prostate concerns; and if the person is a person other than the person with prostate concerns: determining whether the person is subject to chronic retention of urination, in light of measurements, including: frequency of the urination, duration of the urination, volume of the urination, and pressure of urination; and if it is determined that the person is subject to chronic retention of urination, using the camera to capture the identities of the person urinating when the person is leaving the room containing the toilet upon detected by the IR transceiver associated with the door, so that the person can be notified for having prostate irregularities.

In yet another embodiment, the method comprises detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns; if the person is the person with prostate concerns: determining whether the person shows improvements with respect to chronic retention of urination, in light of medicines consumed by the person, and past and current measurements, including: frequency of the urination, duration of the urination, volume of the urination, and the pressure of urination; and if it is determined that the person shows improvements with respect to chronic retention of urination, reporting to a medical center by electronic means, wherein the report contains the medicines and current measurements with respect to the person with prostate concerns; and if the person is a person other than the person with prostate concerns: determining whether the person is subject to chronic retention of urination, in light of measurements, including: frequency of the urination, duration of the urination, volume of the urination, and pressure of urination; and if it is determined that the person is subject to chronic retention of urination, using the camera to capture the identities of the person urinating when the person is leaving the room containing the toilet upon detected by the IR transceiver associated with the door, so that the person can be notified for having prostate irregularities.

In yet another embodied system, the method comprises detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns; if the person is the person with prostate concerns: determining whether the person shows signs of deterioration with respect to chronic retention of urination by using the urine collector attached to the toilet, the pressure sensor, the timer, and the IR transceiver, in light of the substance found in the person's urination streams, and past and current measurements, including: frequency of the urination, duration of the urination, volume of the urination, and the pressure of urination; and if it is determined that the person shows signs of deterioration with respect to chronic retention of urination, reporting to a medical center by electronic means, wherein the report contains the substance found in the person's urination streams and current measurements with respect to the person with prostate concerns; and if the person is a person other than the person with prostate concerns: determining whether the person is subject to chronic retention of urination, in light of the substance found in the person's urination streams and the measurements, including: frequency of the urination, duration of the urination, volume of the urination, and pressure of urination; and if it is determined that the person is subject to chronic retention of urination, using the camera to capture the identities of the person urinating when the person is leaving the room containing the toilet upon detected by the IR transceiver associated with the door, so that the person can be notified for having prostate irregularities.

### BRIEF DESCRIPTION OF THE DRAWINGS

FIG. 1 illustrates an exemplary system to handle prostate-related waste material.

### DETAILED DESCRIPTION

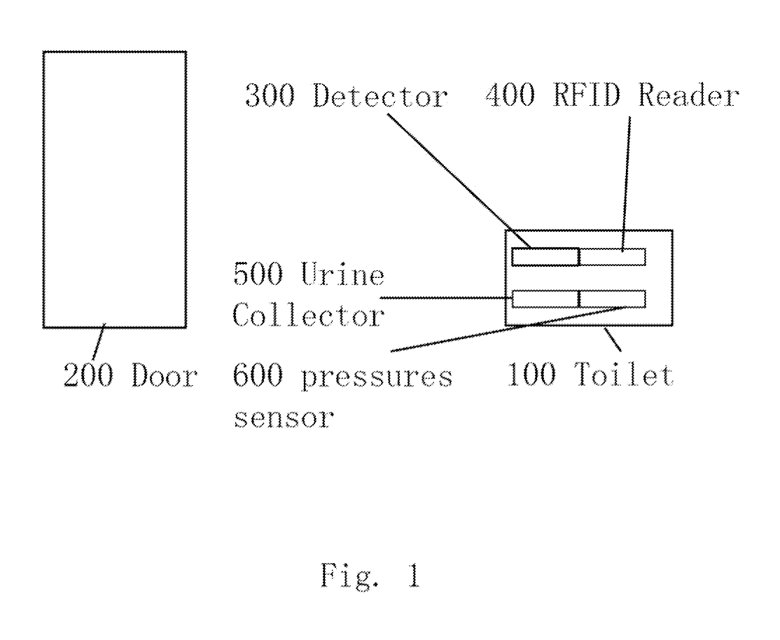

A waste discharging system for aiding patients concerning prostate irregularities is provided. In one implementation, a system is provided. Referring to FIG. 1, a waste discharging system is provided. The system comprises a toilet shared by many persons; a detector detecting that urination occurs; a RFID reader attached to the toilet; a RFID tag associated with a person with prostate concerns; a urine collector attached to the toilet; a pressure sensor associated with the toilet; a timer associated with the toilet; an IR transceiver associated with the toilet; an IR transceiver associated with the door; a camera outside a door of a room containing the toilet, wherein the camera is used to capture identities of persons detected with prostate irregularities but without associating with RFID tag; a processor; and a memory storing instructions that cause the processor to execute a method, wherein the method comprising detecting whether a person urinating is the person with prostate concerns, based on the RFID reader attached to the toilet and the RFID tag associated with the person with prostate concerns; if the person is the person with prostate concerns: determining whether the person has total blockage of urine by using the urine collector attached to the toilet, the pressure sensor, the timer, and the IR transceiver associated with the toilet, in light of measurements, including: frequency of the urination, duration of the urination, volume of the urination, and the pressure of urination; and if it is determined that the total blockage of urine occurs, reporting to a medical center by electronic means, wherein the report contains the measurements with respect to the person with prostate concerns; and if the person is a person other than the person with prostate concerns: determining whether the person has total blockage of urine, in light of measurements, including: frequency of the urination, duration of the urination, volume of the urination, and pressure of urination; and if it is determined that the total blockage of urine occurs, using the camera to capture the identities of the person urinating when the person is leaving the room containing the toilet upon detected by the IR transceiver associated with the door, so that the person can be notified for having prostate irregularities.

In another implementation, a waste discharging system for aiding patients concerning prostate irregularities can comprise a toilet shared by many persons; a detector detecting that urination occurs; a RFID reader attached to the toilet; a RFID tag associated with a person with prostate concerns; a urine collector attached to the toilet; a pressure sensor associated with the toilet; a timer associated with the toilet; an IR transceiver associated with the toilet; an IR transceiver associated with the room; a camera outside a door of a room containing the toilet, wherein the camera is used to capture identities of persons detected with prostate irregularities but without associating with RFID tag; a processor; and a memory storing instructions that cause the processor to execute a method, the method comprising: detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns; if the person is the person with prostate concerns: determining whether the person is subject to chronic retention of urination, in light of measurements, including: frequency of the urination, duration of the urination, volume of the urination, and the pressure of urination; and if it is determined that the person is subject to chronic retention of urination, reporting to a medical center by electronic means, wherein the report contains the measurements with respect to the person with prostate concerns; and if the person is a person other than the person with prostate concerns: determining whether the person is subject to chronic retention of urination, in light of measurements, including: frequency of the urination, duration of the urination, volume of the urination, and pressure of urination; and if it is determined that the person is subject to chronic retention of urination, using the camera to capture the identities of the person urinating when the person is leaving the room containing the toilet upon detected by the IR transceiver associated with the door, so that the person can be notified for having prostate irregularities.

In yet another implementation, a waste discharging system for aiding patients concerning prostate irregularities may comprises a toilet shared by many persons; a urine collector attached to the toilet; a detector detecting that urination occurs; a RFID reader attached to the toilet; a RFID tag associated with a person with prostate concerns; a urine collector attached to the toilet; a pressure sensor associated with the toilet; a timer associated with the toilet; an IR transceiver associated with the toilet; an IR transceiver associated with the room; a camera outside a door of a room containing the toilet, wherein the camera is used to capture identities of persons detected with prostate irregularities but without associating with RFID tag; a processor; a memory storing instructions that cause the processor to execute a method, the method comprising: detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns; if the person is the person with prostate concerns: determining whether the person shows improvements with respect to chronic retention of urination, in light of medicines consumed by the person, and past and current measurements, including: frequency of the urination, duration of the urination, volume of the urination, and the pressure of urination; and if it is determined that the person shows improvements with respect to chronic retention of urination, reporting to a medical center by electronic means, wherein the report contains the medicines and current measurements with respect to the person with prostate concerns; and if the person is a person other than the person with prostate concerns: determining whether the person is subject to chronic retention of urination, in light of measurements, including: frequency of the urination, duration of the urination, volume of the urination, and pressure of urination; and if it is determined that the person is subject to chronic retention of urination, using the camera to capture the identities of the person urinating when the person is leaving the room containing the toilet upon detected by the IR transceiver associated with the door, so that the person can be notified for having prostate irregularities.

Further, the inventive system may comprise a toilet shared by many persons; a urine collector attached to the toilet; a detector detecting that urination occurs; a RFID reader attached to the toilet; a RFID tag associated with a person with prostate concerns; a urine collector attached to the toilet; a pressure sensor associated with the room; a timer associated with the room; an IR transceiver associated with the room; an IR transceiver associated with the door; a urine sensor for detecting whether a substance, including alcohol and caffeine, has been found in urination streams; a camera outside a door of a room containing the toilet, wherein the camera is used to capture identities of persons detected with prostate irregularities but without associating with RFID tag; a processor; and a memory storing instructions that cause the processor to execute a method, the method comprising: detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns; if the person is the person with prostate concerns: determining whether the person shows signs of deterioration with respect to chronic retention of urination by using the urine collector attached to the toilet, the pressure sensor, the timer, and the IR transceiver, in light of the substance found in the person's urination streams, and past and current measurements, including: frequency of the urination, duration of the urination, volume of the urination, and the pressure of urination; and if it is determined that the person shows signs of deterioration with respect to chronic retention of urination, reporting to a medical center by electronic means, wherein the report contains the substance found in the person's urination streams and current measurements with respect to the person with prostate concerns; and if the person is a person other than the person with prostate concerns: determining whether the person is subject to chronic retention of urination, in light of the substance found in the person's urination streams and the measurements, including: frequency of the urination, duration of the urination, volume of the urination, and pressure of urination; and if it is determined that the person is subject to chronic retention of urination, using the camera to capture the identities of the person urinating when the person is leaving the room containing the toilet upon detected by the IR transceiver associated with the door, so that the person can be notified for having prostate irregularities.

What is claimed is:

1. A waste discharging system for aiding patients concerning prostate irregularities, comprising:
a toilet shared by many persons;
a detector detecting that urination occurs;
a RFID reader attached to the toilet;
a RFID tag associated with a person with prostate concerns;
a urine collector attached to the toilet;
a pressure sensor associated with the toilet;
a timer associated with the toilet;
an IR transceiver associated with the toilet;
an IR transceiver associated with the door;
a camera outside a door of a room containing the toilet, wherein the camera is used to capture identities of persons detected with prostate irregularities but without associating with RFID tag;
a processor; and
a memory storing instructions that cause the processor to execute a method.

2. The system of claim 1, wherein the method comprises:
detecting whether a person urinating is the person with prostate concerns, based on the RFID reader attached to the toilet and the RFID tag associated with the person with prostate concerns;
if the person is the person with prostate concerns:
determining whether the person has total blockage of urine by using the urine collector attached to the toilet, the pressure sensor, the timer, and the IR transceiver associated with the toilet, in light of measurements, including: frequency of the urination, duration of the urination, volume of the urination, and the pressure of urination; and
if it is determined that the total blockage of urine occurs, reporting to a medical center by electronic means, wherein the report contains the measurements with respect to the person with prostate concerns; and
if the person is a person other than the person with prostate concerns:
determining whether the person has total blockage of urine, in light of measurements, including: frequency of the urination, duration of the urination, volume of the urination, and pressure of urination; and
if it is determined that the total blockage of urine occurs, using the camera to capture the identities of the person urinating when the person is leaving the room containing the toilet upon detected by the IR transceiver associated with the door, so that the person can be notified for having prostate irregularities.

3. The system of claim 1, wherein the method comprises:
detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns;
if the person is the person with prostate concerns:
determining whether the person is subject to chronic retention of urination, in light of measurements, including: frequency of the urination, duration of the urination, volume of the urination, and the pressure of urination; and
if it is determined that the person is subject to chronic retention of urination, reporting to a medical center by electronic means, wherein the report contains the measurements with respect to the person with prostate concerns; and
if the person is a person other than the person with prostate concerns:
determining whether the person is subject to chronic retention of urination, in light of measurements, including: frequency of the urination, duration of the urination, volume of the urination, and pressure of urination; and
if it is determined that the person is subject to chronic retention of urination, using the camera to capture the identities of the person urinating when the person is leaving the room containing the toilet upon detected by the IR transceiver associated with the door, so that the person can be notified for having prostate irregularities.

4. The system of claim 1, wherein the method comprises:
detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns;
if the person is the person with prostate concerns:
determining whether the person shows improvements with respect to chronic retention of urination, in light of medicines consumed by the person, and past and current measurements, including: frequency of the urination, duration of the urination, volume of the urination, and the pressure of urination; and
if it is determined that the person shows improvements with respect to chronic retention of urination, reporting to a medical center by electronic means, wherein the report contains the medicines and current measurements with respect to the person with prostate concerns; and
if the person is a person other than the person with prostate concerns:
determining whether the person is subject to chronic retention of urination, in light of measurements, including: frequency of the urination, duration of the urination, volume of the urination, and pressure of urination; and
if it is determined that the person is subject to chronic retention of urination, using the camera to capture the identities of the person urinating when the person is leaving the room containing the toilet upon detected by the IR transceiver associated with the door, so that the person can be notified for having prostate irregularities.

5. The system of claim 1, wherein the method comprises:
detecting whether a person urinating is the person with prostate concerns by using the urine collector attached to the toilet, the pressure sensor, the timer, and the IR transceiver associated with the toilet, based on the RFID reader attached to the toilet and the RFID tag associated with the person with prostate concerns;
if the person is the person with prostate concerns:
determining whether the person shows signs of deterioration with respect to chronic retention of urination by using the urine collector attached to the toilet, the pressure sensor, the timer, and the IR transceiver, in light of the substance found in the person's urination streams, and past and current measurements, including: frequency of the urination, duration of the urination, volume of the urination, and the pressure of urination; and
if it is determined that the person shows signs of deterioration with respect to chronic retention of urination, reporting to a medical center by electronic means, wherein the report contains the substance found in the person's urination streams and current measurements with respect to the person with prostate concerns; and
if the person is a person other than the person with prostate concerns:
determining whether the person is subject to chronic retention of urination, in light of the substance found in the person's urination streams and the measurements, including: frequency of the urination, duration of the urination, volume of the urination, and pressure of urination; and
if it is determined that the person is subject to chronic retention of urination, using the camera to capture the identities of the person urinating when the person is leaving the room containing the toilet upon detected by the IR transceiver associated with the door, so that the person can be notified for having prostate irregularities.
